(19) United States
(12) Patent Application Publication
(10) Pub. No.: US 2015/0335567 A1
(43) Pub. Date: Nov. 26, 2015

(54) NOVEL OIL-IN-WATER EMULSIONS RICH IN SALTS, HAVING HIGH VISCOSITY AND STABLE OVER TIME

(71) Applicant: SOCIETE D'EXPLOITATION DE PRODUITS POUR LES INDUSTRIES CHIMIQUES SEPPIC, Paris (FR)

(72) Inventor: Emmanuelle MERAT, Lautrec (FR)

(73) Assignee: SOCIETE D'EXPLOITATION DE PRODUITS POUR LES INDUSTRIES CHIMIQUES SEPPIC, PARIS (FR)

(21) Appl. No.: 14/758,811

(22) PCT Filed: Nov. 26, 2013
(86) PCT No.: PCT/FR2013/052857

§ 371 Date: Jul. 1, 2015

(30) Foreign Application Priority Data
Jan. 4, 2013 (FR) 1350065

(51) Int. Cl. A61K 8/81; A61Q 17/04; A61K 8/73; A61Q 19/00; A61K 8/06; A61K 8/92

(52) U.S. Cl. CPC A61K 8/8194; A61K 8/062; A61K 8/92; A61K 8/737; A61Q 19/00; A61Q 17/04; A61K 2800/594; A61K 2800/48

(57) ABSTRACT

A composition in the form of an oil-in-water emulsion includes, for 100% of its mass: from 5%-55% by mass of a fatty phase consisting of at least one oil and/or at least one wax, from 0.025% to 3.75% of at least one cross-linked anionic polyelectrolyte resulting from the polymerisation of at least one monomer having a strong acid function, partially or completely salified, with at least one neutral monomer, and at least one monomer of formula (I), in which R represents a linear or branched alkyl radical including from eight to twenty carbon atoms and n represents a number greater than or equal to one and less than or equal to twenty, in the presence of at least one crosslinking agent, from 0.025% to 3.75% of at least one galactomannan having a degree of substitution of approximately 1/1 or of approximately ½ or of approximately ⅕.

The invention relates to novel oil-in-water emulsions, as well as uses thereof in cosmetics and in pharmacy.

Cosmetic compositions in the form of oil-in-water emulsions marketed by the cosmetics industry and by the pharmaceutical industry very regularly comprise synthetic polymer thickeners for increasing the viscosity of said oil-in-water emulsions, which may be in the form of creams and of milks, and which are applied directly on the skin.

These synthetic polymer thickeners make it possible to thicken the aqueous phases present in said oil-in-water emulsions, thus providing either the desired consistency or an effect of stabilization of said emulsion.

The synthetic polymer thickeners currently used in these fields are in two physical forms, the powder form and the liquid form for which the polymer is prepared by radical polymerization in an inverted emulsion using surfactants, and which is commonly called an inverse latex.

Among the best known synthetic polymer thickeners in powder form, we may mention polymers based on acrylic acid or copolymers based on acrylic acid and acrylic esters. We may mention for example the polymers marketed under the brand names CARBOPOL™ and PEMULEN™. They are notably described in the American patents U.S. Pat. No. 5,373,044, U.S. Pat. No. 2,798,053 and in European patent EP 0 301 532.

In cosmetics, homopolymers or copolymers based on 2-acrylamido-2-methyl-propanesulfonic acid and/or salts thereof are also used, always in the form of powder.

These polymer thickeners are marketed under the brand name Aristoflex™ and are notably described in European patents EP 816 403, EP 1 116 733 and EP 1 069 142. These synthetic thickeners in powder form are obtained by precipitation polymerization; the monomer(s) is/are dissolved in an organic solvent of the benzene, ethyl acetate, cyclohexane, or tert-butanol type; this method therefore requires a great many successive steps of purification of the end product, to remove all traces of residual solvent.

The cosmetic and pharmacy industries also make wide use of thickeners that are in the form of inverse latices and notably those marketed by the applicant. We may mention for example, the thickeners Sepigel™ 305, Simulgel™ 600, Simulgel™ EG, Simulgel™ EPG, Simulgel™ NS, Simulgel™ A, Sepiplus™ 400, Sepiplus™ 250 and Sepiplus™ 265. These thickeners are obtained by radical polymerization in an inverted emulsion. They have the advantage that they are easier to manipulate, notably at room temperature, and they disperse in water very quickly. Moreover, these products develop remarkably high thickening performance; this performance is probably due to the method employed for preparing them: a reaction of dispersed-phase radical polymerization, which leads to polymers with very high molecular weights.

However, these synthetic thickeners that are in the form of an inverse latex contain an oil, and one or more surfactants, which may sometimes induce skin intolerance reactions in subjects who are particularly sensitive; moreover, this presence of oil makes them unusable for preparing clear aqueous gels.

The applicant has therefore developed synthetic thickeners that have thickening performance equivalent to or better than the inverse latices, but are better tolerated by the skin, in particular owing to the absence of any oil phase, which may lead to clearer aqueous gels. These products are in the form of powder but possess dissolution times, and therefore ease of use, comparable to those of products that are in liquid form. These compounds, described in the European patent application published under the number EP 1 496 081, are obtained by the classical polymerization techniques, such as dispersed-phase radical polymerization, inverted-suspension radical polymerization, inverted-emulsion or inverted-microemulsion radical polymerization. The synthetic thickener systems obtained are then extracted and purified by various techniques such as precipitation in a third solvent, precipitation in a third solvent optionally followed by washing, spray-drying or azeotropic dehydration, optionally followed by washing with a carefully selected solvent. These synthetic thickeners therefore combine some of the advantages of the classical synthetic thickeners in the form of powders (absence of oil, production of clearer aqueous gels) and the advantages of the synthetic thickeners in the form of an inverse latex (high dissolution rate, remarkable thickening power and stabilizing properties). However, for certain uses, the customers who use these synthetic thickener systems wish to be able to manufacture gels that are even clearer than those obtained today, or even transparent gels. Moreover, the gels obtained with these synthetic thickeners do not have satisfactory stability when the composition is rich in electrolytes, as is often the case with compositions comprising sun filters and/or colored pigments and/or plant extracts rich in electrolytes.

The applicant has also developed synthetic thickener systems such as those described in the French patent application published under the number 2 910 899, which discloses a linear, branched or crosslinked terpolymer of at least one monomer possessing a free, partially salified or totally salified strong acid function, with at least one neutral monomer, and at least one monomer of formula (A):

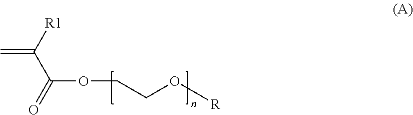

in which R1 represents a hydrogen atom or a methyl radical, R represents a linear or branched alkyl radical comprising from eight to thirty carbon atoms and n represents a number greater than or equal to one and less than or equal to fifty. These polymers have very pronounced thickening properties, notably in the presence of electrolytes. They function over a wide range of pH and make it possible to produce transparent gels. However, formulations with low pH thickened with certain of them do not have satisfactory long-term stability in the presence of salts and some of them, which contain fatty alcohols, have a rather unattractive elastic appearance and produce sensations of stickiness to the touch and/or an appearance of a discontinuous cream or grainy emulsion.

The applicant has demonstrated that these drawbacks could be avoided by selecting certain of these terpolymers, which were not disclosed in the French patent application published under the number 2 910 899, and has developed novel branched or crosslinked anionic polyelectrolytes, such as those described in the international application published under the number WO 2011/030044, which result from radical polymerization of at least one monomer possessing a partially salified or totally salified strong acid function, with at least one neutral monomer, and at least one monomer of formula (B):

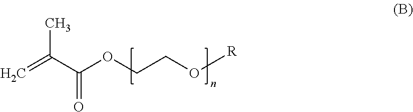

in which R represents a linear or branched alkyl radical comprising from eight to twenty carbon atoms and n represents a number greater than or equal to one and less than or equal to thirty.

The cosmetic and pharmacy industries are also looking for galenical forms that reduce the risks of cutaneous intolerance, and consequently tend to select ingredients as constituents of said galenical forms that are well tolerated, and in addition tend to reduce the relative proportion of ingredients that are likely to increase the probability of reactions of intolerance on the skin. In this respect, the cosmetic and pharmacy industries are endeavouring to develop oil-in-water emulsions that do not contain stabilizer systems comprising emulsifying surfactants. The terpolymers described in the international application published under the number WO 2011/030044 therefore constitute ideal candidates for preparing oil-in-water emulsions free from emulsifying surfactants.

However, when oil-in-water emulsions are prepared using these synthetic thickener terpolymers in the absence of emulsifying surfactants, appearance of lumps is observed in said oil-in-water emulsions free from emulsifying surfactants during storage thereof over time. It is therefore necessary to develop new oil-in-water emulsions, free from emulsifying surfactants, that do not form lumps during prolonged storage, but retain high viscosity in the presence of media rich in electrolytes and over a wide range of pH, as well as satisfactory sensory properties, namely without a sticky and ropy character when handled and after application on the skin.

Polysaccharides have been in use for many years as agents for modifying texture and/or rheology for preparing food, cosmetic or pharmaceutical compositions. Depending on their chemical constitution, they may be used as gelling agents and/or as thickeners. “Thickener” means a chemical compound that increases the viscosity of the medium into which it is introduced. “Gelling agent” means a compound that transforms a liquid medium into a structured state, which does not flow, by forming a three-dimensional network within the liquid; gel being regarded as an intermediate state between the liquid state and the solid state.

The polysaccharides are polymers of saccharides. The IUPAC definition of the saccharides designates monosaccharides, compounds of monosaccharides proper and their derivatives obtained either by reduction of a carbonyl group, or by oxidation of one or more hydroxyl functions, or by the replacement of one or more hydroxyl functions with a hydrogen atom, an amine group, a phosphate function, or a sulfate function.

The polysaccharides most commonly used for preparing food, cosmetic or pharmaceutical compositions mainly consist of monosaccharides, such as glucose, galactose, mannose or of derivatives of monosaccharides for which the hydroxyl function of the terminal carbon has been oxidized to a carboxyl function. Consequently, we may distinguish two distinct groups among the polysaccharides: the polysaccharides consisting solely of monosaccharides (or “poly-monosaccharides”) and the polysaccharides consisting of derivatives of monosaccharides. Among the polysaccharides made up solely of monosaccharides, we may distinguish:
glucans, which are homopolymers of glucose and are very abundant in nature,
glucomannoglycans,
xyloglycans,
galactomannans, which are polymers whose main chain consists of D-mannose units, joined together at β-1,4, and on which D-galactose units are grafted laterally by α-1,6 bonds.

Galactomannans occur in several plant species, and more particularly in the leguminous species, in which they constitute the albumen of the seeds.

Depending on the plant origin from which the galactomannans are derived, the degree of substitution (DS) of the D-galactose units on the main chain of D-mannose varies between 0 and 1:
galactomannans derived from cassia gum have a degree of substitution (DS) of about ⅕, signifying lateral grafting of one unit of D-galactose to every 5 D-mannose units present on the main chain of the polysaccharide,
galactomannans derived from carob gum have a degree of substitution (DS) of about ¼, signifying lateral grafting of one unit of D-galactose to every 4 D-mannose units present on the main chain of the polysaccharide,
galactomannans derived from tara gum have a degree of substitution (DS) of about ⅓, signifying lateral grafting of one unit of D-galactose to every 3 D-mannose units present on the main chain of the polysaccharide,
galactomannans derived from guar gum have a degree of substitution (DS) of about ½, signifying lateral grafting of one unit of D-galactose to every 2 D-mannose units present on the main chain of the polysaccharide,
galactomannans derived from fenugreek gum have a degree of substitution (DS) of about 1/1, signifying lateral grafting of one unit of D-galactose for nearly all the D-mannose units present on the main chain of the polysaccharide.

As a subcategory of the polysaccharides, galactomannans have already been combined with synthetic thickeners resulting from the radical polymerization of monomers such as acrylic acid, esters of acrylic acid, 2-acrylamido-2-methyl-propanesulfonic acid and/or salts thereof, acrylamide, 2-hydroxyethyl acrylate.

The US patent published under the number U.S. Pat. No. 4,540,510 describes the preparation of aqueous gels using homopolymers of 2-acrylamido-2-methyl-propanesulfonic acid and galactomannans, such as those derived from guar gum and tara gum. However, the aqueous gels that are obtained and used for applications in the field of fracturing of underground cavities, or preparation of pigment pastes for printing on textiles, or suspension of pigments in paints, or in the preparation of cosmetic formulations comprising alcohols as co-solvents, do not allow attainment of viscosity levels sufficient to allow the preparation of oil-in-water emulsions rich in electrolytes and stable in storage.

The French patent application published under the number 2 940 111 describes the use of compositions comprising polysaccharides, which may be combined with hydrophilic gelling agents notably selected from copolymers comprising 2-acrylamido-2-methyl-propanesulfonic acid and acrylamide as constituent monomers, or comprising 2-acrylamido-2-methylpropanesulfonic acid and methacrylates of polyethoxylated alkyls. These compositions are intended for uses in makeup, possessing the property of not transferring to the substrates with which they are brought into contact, as well as the property of water resistance after application on the skin. However, the hydrophilic gelling agents described in the French patent application published under the number 2 940 111 are known for not allowing high levels of viscosity to be attained in the presence of media rich in electrolytes.

The inventors therefore tried to develop new oil-in-water emulsions, free from emulsifying surfactants in their stabilizing system, rich in salts, maintaining high viscosity and a homogeneous appearance after prolonged storage.

That is why according to a first aspect, the invention relates to a composition (C₁) that is in the form of an emulsion of the oil-in-water type, characterized in that it comprises for 100% of its weight:
from 5 to 55 wt %, more particularly from 7 to 30 wt % and even more particularly from 10 to 20 wt % of a fatty phase (P₁) consisting of at least one oil and/or of at least one wax,
from 0.025 to 3.75 wt %, more particularly from 0.125% to 3%, and even more particularly from 0.125% to 2.25% of at least one crosslinked anionic polyelectrolyte (AP) resulting from the polymerization of at least one monomer possessing a partially or totally salified strong acid function, with at least one neutral monomer, and at least one monomer of formula (I):

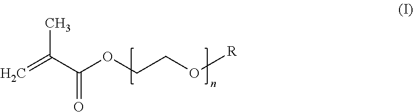

in which R represents a linear or branched alkyl radical comprising from eight to twenty carbon atoms and n represents a number greater than or equal to one and less than or equal to twenty, in the presence of at least one crosslinking agent,
from 0.025 to 3.75 wt %, more particularly from 0.125 to 3 wt % and even more particularly from 0.125 to 2.25 wt % of at least one galactomannan (GM) having a degree of substitution (DS) of about 1/1 or of about ½ or of about ¼ or of about ⅕,
from 37.5 to 94.95 wt %, more particularly from 55 to 94.95 wt %, and even more particularly from 65 to 94.95 wt % of a cosmetically acceptable aqueous phase (P₂), said aqueous phase (P₂) comprising for 100% of its weight, from 1 to 25 wt %, more particularly from 1.5 to 20 wt %, and even more particularly from 2 to 10 wt % of at least one salt (S) in dissolved form,
   said composition (C₁) being further characterized in that the weight ratio of the galactomannan (GM) to the crosslinked anionic polyelectrolyte (AP) is greater than or equal to ⅓ and less than or equal to 3/1, more particularly greater than or equal to ½ and less than or equal to 3/2, and even more particularly greater than or equal to ⅔ and less than or equal to 1.

“Oils” means, in the present application, compounds and/or mixtures of compounds that are insoluble in water, which are in the liquid state at a temperature of 25° C. Among the oils usable in the fatty phase (P₁) of composition (C₁) according to the present invention, we may mention:
mineral oils such as paraffin oil, liquid paraffin, isoparaffins or mineral white oils;
oils of animal origin, such as squalene or squalane;
vegetable oils, such as phytosqualane, sweet almond oil, copra oil, castor oil, jojoba oil, olive oil, colza oil, peanut oil, sunflower oil, wheat germ oil, maize germ oil, soybean oil, cottonseed oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils obtained from flowers or from vegetables;
ethoxylated vegetable oils;
synthetic oils such as fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, esters derived from lanolin acid, such as isopropyl lanolate, isocetyl lanolate, monoglycerides, diglycerides and triglycerides of fatty acids such as glycerol triheptanoate, alkylbenzoates, hydrogenated oils, poly(alpha-olefin), the polyolefins such as poly(isobutane), the synthetic isoalkanes such as isohexadecane, isododecane, perfluorinated oils; and
silicone oils such as dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy modified silicones, silicones modified with fluorinated groups, cyclic silicones and silicones modified with alkyl groups.

“Waxes” means, in the present application, compounds and/or mixtures of compounds that are insoluble in water, and are solid at a temperature greater than or equal to 45° C. Among the waxes usable in the fatty phase (P₁) of composition (C₁) according to the present invention, we may mention beeswax, carnauba wax, candelilla wax, ouricury wax, Japan wax, cork fiber wax, sugar cane wax, paraffin waxes, lignite waxes, microcrystalline waxes, lanolin wax; ozokerite; polyethylene wax; silicone waxes; vegetable waxes; fatty alcohols and fatty acids that are solid at room temperature; glycerides that are solid at room temperature.

Among the other fats that may be combined with the fatty phase (P₁) of composition (C₁) according to the present invention, we may mention the linear or branched, saturated or unsaturated fatty alcohols, or the linear or branched, saturated or unsaturated fatty acids.

In the composition (C₁) according to the present invention, “a galactomannan (GM) having a degree of substitution (DS) of about ⅕” denotes a polysaccharide whose main chain consists of D-mannose units, joined together in the β-1,4 position, and on which D-galactose units are grafted laterally with α-1,6 bonds, in such a way that grafting of a D-galactose unit is observed on average every 5 D-mannose units present on the main chain of the polysaccharide. The galactomannan (GM) as defined above is derived more particularly from cassia gum.

In the composition (C₁) according to the present invention, “a galactomannan (GM) having a degree of substitution (DS) of about ¼” denotes a polysaccharide whose main chain consists of D-mannose units, joined together in the β-1,4 position, and on which D-galactose units are grafted laterally with α-1,6 bonds, in such a way that grafting of a D-galactose unit is observed on average every 4 D-mannose units present on the main chain of the polysaccharide. The galactomannan (GM) as defined above is derived more particularly from carob gum.

In the composition (C₁) according to the present invention, “a galactomannan (GM) having a degree of substitution (DS) of about ½” denotes a polysaccharide whose main chain consists of D-mannose units, joined together in the β-1,4 position, and on which D-galactose units are grafted laterally with α-1,6 bonds, in such a way that grafting of a D-galactose unit is observed on average every 2 D-mannose units present on the main chain of the polysaccharide. The galactomannan (GM) as defined above is derived more particularly from guar gum.

In the composition (C₁) according to the present invention, “a galactomannan (GM) having a degree of substitution (DS) of about 1/1” denotes a polysaccharide whose main chain consists of D-mannose units, joined together in the β-1,4 position, and on which D-galactose units are grafted laterally with α-1,6 bonds, in such a way that grafting of a D-galactose unit is observed on average every D-mannose unit present on the main chain of the polysaccharide. The galactomannan (GM) as defined above is derived more particularly from fenugreek gum.

According to a particular aspect of the present invention, the composition (C₁) as defined above is characterized in that said galactomannan (GM) has a degree of substitution (DS) of about ½.

“Crosslinked anionic polyelectrolyte (AP)” denotes, in the definition of composition (C₁) according to the present invention, a crosslinked nonlinear anionic polyelectrolyte, which is in the state of a three-dimensional network that is insoluble in water, but is swellable with water and leads to the production of a chemical gel. “Partially salified or totally salified” signifies, in the definition of the crosslinked anionic polyelectrolyte (AP) present in composition (C₁) as defined above, that said strong acid function of the monomer bearing it is partially or totally salified, generally in the form of an alkali metal salt, for example a sodium salt or a potassium salt, or in the form of an ammonium salt.

In the composition (C₁) as defined above according to the present invention, said crosslinked anionic polyelectrolyte (AP) as defined above generally comprises between 5 mol % and 95 mol % of monomers with a strong acid function, more particularly between 10 mol % and 90 mol %, and quite particularly between 20 mol % and 80 mol %.

In the composition (C₁) as defined above according to the present invention, said crosslinked anionic polyelectrolyte (AP) as defined above generally comprises between 4.9 mol % and 90 mol % of a neutral monomer, more particularly between 9.5 mol % and 85 mol %, and quite particularly between 15 mol % and 75 mol %.

In the composition (C₁) as defined above according to the present invention, said crosslinked anionic polyelectrolyte (AP) as defined above generally comprises between 0.1 mol % and 10 mol % of monomers of formula (I) and more particularly between 0.5 mol % and 5 mol %.

In the composition (C₁) as defined above according to the present invention, in said crosslinked anionic polyelectrolyte (AP) as defined above, the strong acid function of said monomer bearing it is notably the sulfonic acid function, partially or totally salified. In the composition (C₁) as defined above according to the present invention, in said crosslinked anionic polyelectrolyte (AP) as defined above, the neutral monomer is notably selected from acrylamide, methacrylamide, N-alkylacrylamide, in which the alkyl group comprises from one to four carbon atoms, for example N-methylacrylamide, N-ethylacrylamide, N-propylacrylamide, N-isopropylacrylamide, N-butylacrylamide, N-(tert-butyl)acrylamide, N-alkylmethacrylamide, in which the alkyl group comprises from one to four carbon atoms, for example N-methylmethacrylamide, N-ethylmethacrylamide, N-propylmethacrylamide, N-isopropylmethacrylamide, N-butylmethacrylamide or N-(tert-butyl)methacrylamide, N,N-dialkylacrylamide, in which each of the alkyl groups comprises between one and four carbon atoms, for example N,N-dimethylacrylamide, N,N-diethylacrylamide, N,N-dipropylacrylamide, (2-hydroxyethyl)acrylate, (2,3-dihydroxypropyl)acrylate, (2-hydroxyethyl)methacrylate, (2,3-dihydroxypropyl)methacrylate, diacetone acrylamide or an ethoxylated derivative of molecular weight between 400 g/mol and 1000 g/mol of each of these esters, or vinylpyrrolidone.

In formula (I) as defined above according to the present invention, “linear or branched alkyl radical comprising from eight to twenty carbon atoms” denotes more particularly for R:
either a radical derived from the linear primary alcohols such as for example the octyl, decyl, undecyl, dodecyl, tridecyl, tetradecyl, pentadecyl, hexadecyl, heptadecyl, octadecyl, nonadecyl or eicosyl radical;
or else a radical derived from the Guerbet alcohols, which are branched 1-alkanols corresponding to the general formula:

CH₃—(CH₂)ₚ—CH[CH₃—(CH₂)ₚ₋₂]—CH₂OH,

in which ρ represents an integer between 2 and 9, such as for example the 2-ethylhexyl, 2-propylheptyl, 2-butyloctyl, 2-pentylnonyl, 2-hexyldecyl or 2-octyldodecyl radical;
or else a radical derived from the isoalkanols corresponding to the general formula:

CH₃—CH(CH₃)—(CH₂)ₘ—CH₂OH,

in which m represents an integer between 2 and 16, such as for example the 4-methylpentyl, 5-methylhexyl, 6-methylheptyl, 15-methylpentadecyl or 16-methylheptadecyl radical, or the 2-hexyloctyl, 2-octyldecyl or 2-hexyldodecyl radical.

According to a particular aspect, the invention relates to a composition (C₁) as defined above, characterized in that said crosslinked anionic polyelectrolyte (AP) comprises for 100 mol % of its constituent monomers:
from 20 mol % to 80 mol % of monomer units derived from a monomer bearing a partially or totally salified strong acid function;
from 15 mol % to 75 mol % of monomer units derived from a neutral monomer;
from 0.5 to 5 mol % of monomer units derived from a monomer of formula (I).

According to another particular aspect, the invention relates to a composition (C₁) as defined above, characterized in that in said crosslinked anionic polyelectrolyte (AP), said monomer possessing a strong acid function is 2-methyl 2-[(1-oxo 2-propenyl)amino] 1-propanesulfonic acid, partially salified or totally salified, and more particularly 2-methyl 2-[(1-oxo 2-propenyl)amino] 1-propanesulfonic acid partially salified or totally salified in the form of an alkali metal salt, for example a sodium salt or a potassium salt, or in the form of an ammonium salt.

According to another particular aspect, the invention relates to a composition (C₁) as defined above, characterized in that in said crosslinked anionic polyelectrolyte (AP), said neutral monomer is selected from acrylamide, (2-hydroxyethyl)acrylate or N,N-dimethylacrylamide.

According to a particular aspect of the present invention, the latter relates to a composition (C₁) as defined above, characterized in that in said crosslinked anionic polyelectrolyte (AP) and for said monomer of formula (I) as defined above, R represents an alkyl radical comprising from 12 to 18 carbon atoms.

According to another particular aspect, the invention relates to a composition (C₁) as defined above, characterized in that in said crosslinked anionic polyelectrolyte (AP) and for said monomer of formula (I) as defined above, n represents an integer between 3 and 20.

According to an even more particular aspect, the invention relates to a composition (C₁) as defined above, characterized in that in said crosslinked anionic polyelectrolyte (AP), said monomer of formula (I) is tetraethoxylated lauryl methacrylate.

According to an even more particular aspect, the invention relates to a composition (C₁) as defined above, characterized in that the monomer of formula (I) comprised in the crosslinked anionic polyelectrolyte (AP) is eicosaethoxylated stearyl methacrylate.

According to an even more particular aspect, the invention relates to a composition (C₁) as defined above, characterized in that the monomer of formula (I) comprised in the crosslinked anionic polyelectrolyte (AP) is behenyl methacrylate ethoxylated with 25 moles of ethylene oxide.

According to another particular aspect, the invention relates to a composition (C₁) as defined above, for which said crosslinked anionic polyelectrolyte (AP) is crosslinked with a diethylenic or polyethylenic compound in the molar proportion, expressed relative to the monomers employed, from 0.005 to 1%, more particularly from 0.01 to 0.5% and quite particularly from 0.01 to 0.25%. The crosslinking agent is more particularly selected from ethylene glycol dimethacrylate, tetraallyloxyethane, ethylene glycol diacrylate, diallyl urea, triallyl amine, trimethylol propanetriacrylate or methylene-bis(acrylamide) or a mixture of these compounds.

The crosslinked anionic polyelectrolyte (AP) employed in the composition (C₁) as defined above, according to the present invention, may also comprise various additives, such as complexing agents, transfer agents or chain-limiting agents.

According to a particular aspect, the invention relates to a composition (C₁) as described above in which said crosslinked anionic polyelectrolyte (AP) is selected from the terpolymers of 2-methyl 2-[(1-oxo 2-propenyl)amino] 1-propanesulfonic acid partially salified in the form of ammonium salt, acrylamide and tetraethoxylated lauryl methacrylate, crosslinked with trimethylol propanetriacrylate, the terpolymers of 2-methyl 2-[(1-oxo 2-propenyl)amino] 1-propanesulfonic acid partially salified in the form of ammonium salt, acrylamide and eicosaethoxylated stearyl methacrylate, crosslinked with trimethylol propanetriacrylate, the terpolymers of 2-methyl 2-[(1-oxo 2-propenyl)amino] 1-propanesulfonic acid partially salified in the form of ammonium salt, 2-hydroxyethyl acrylate and tetraethoxylated lauryl methacrylate, crosslinked with trimethylol propanetriacrylate, the terpolymers of 2-5 methyl 2-[(1-oxo 2-propenyl)amino] 1-propanesulfonic acid partially salified in the form of ammonium salt, 2-hydroxyethyl acrylate and eicosaethoxylated stearyl methacrylate, crosslinked with trimethylol propanetriacrylate, the terpolymers of 2-methyl 2-[(1-oxo 2-propenyl)amino] 1-propanesulfonic acid partially salified in the form of ammonium, N,N-dimethylacrylamide and tetraethoxylated lauryl methacrylate, crosslinked with trimethylol propanetriacrylate or the terpolymers of 2-methyl 2-[(1-oxo 2-propenyl)amino] 1-propanesulfonic acid partially salified in the form of ammonium salt, N,N-dimethylacrylamide and eicosaethoxylated stearyl methacrylate, crosslinked with trimethylol propanetriacrylate.

According to an even more particular aspect, the invention relates to a composition (C₁) as described above, characterized in that said crosslinked anionic polyelectrolyte (AP) is a terpolymer of 2-methyl 2-[(1-oxo 2-propenyl)amino] 1-propanesulfonic acid partially salified in the form of ammonium salt, N,N-dimethylacrylamide and tetraethoxylated lauryl methacrylate, crosslinked with trimethylol propanetriacrylate.

In the composition (C₁) according to the invention, the combination in situ of the crosslinked anionic polyelectrolyte (AP) and galactomannan (GM) as defined above, and in the proportions as defined above, constitutes the stabilizing system of said composition (C₁).

The expression “cosmetically acceptable” used in the definition of the aqueous phase (P₂) of composition (C₁) according to the present invention signifies, according to the directive of the Council of the European Economic Community No. 76/768/EEC of 27 Jul. 1976 as amended by directive No. 93/35/EEC of 14 Jun. 1993, that said aqueous phase (P₂) comprises water and any substance or preparation intended to be brought into contact with the various parts of the human body (epidermis, body hair and hair system, nails, lips and genitalia) or with the teeth and the oral mucosae with a view, exclusively and principally, to clean them, perfume them, alter their appearance and/or to correct body odors from them and/or to protect them or maintain them in a good state.

A cosmetically acceptable aqueous phase (P₂) comprised in the composition (C₁) according to the present invention contains water, and may contain conventionally one or more cosmetically acceptable organic solvents, a mixture of water and of one or more cosmetically acceptable organic solvents. The cosmetically acceptable solvents may more particularly be selected from the polyhydric alcohols, for example glycerol, diglycerol, triglycerol, the oligomers of glycerol, xylitol, erythritol, sorbitol, methyl-2,-propanediol-1,3; the alkoxylated polyhydric alcohols; the glycols, for example butylene glycol, hexylene glycol, caprylyl glycol or 1,2-octanediol or 1,2-pentanediol, pentylene glycol, monopropylene glycol, dipropylene glycol, isoprene glycol, butyldiglycol, the polyethylene glycols whose molecular weight is between 200 g·mol-1 and 8000 g·mol-1; or the water-soluble alcohols, for example ethanol, isopropanol or butanol.

In the composition (C₁) as defined above according to the present invention, “salt (S)” denotes a heteropolar compound whose crystal lattice comprises the participation of at least one type of cation different from the hydrogen ions and of at least one type of anion different from the hydroxide ions.

According to a particular aspect, the salt (S) in dissolved form in the aqueous phase (P₂) of composition (C₁) according to the present invention is selected from the inorganic salts and from the organic salts.

According to this particular aspect, the salt (S) is in particular selected from the inorganic salts.

According to a more particular aspect, the invention relates to a composition (C₁) as defined above, characterized in that the salt (S) is an inorganic salt consisting of a cation, which is the ammonium ion or a metal cation, and of an anion selected from the elements of the group consisting of the halides, carbonates, bicarbonates, phosphates, nitrates, borates and sulfates.

According to a more particular aspect, the invention relates to a composition (C₁) as defined above, characterized in that the salt (S) is an inorganic salt whose metal cation is a monovalent or multivalent cation selected from the elements of the group consisting of the cations of sodium, potassium, lithium, calcium, magnesium, zinc, manganese, iron, copper, cobalt, silver, gold, aluminum, barium, bismuth, selenium, zirconium, strontium and tin.

According to an even more particular aspect, the invention relates to a composition (C1) as defined above, characterized in that the salt (S) is an inorganic salt selected from the elements of the group consisting of sodium chloride, calcium chloride, magnesium chloride, calcium sulfate, ammonium sulfate, calcium carbonate, zinc sulfate, magnesium sulfate, sodium borate.

According to another particular aspect, the salt (S) is in particular selected from the organic salts.

According to a particular aspect, the invention relates to a composition (C₁) as defined above, characterized in that the salt (S) is an organic salt consisting of a cation, which is the ammonium ion or a metal cation, and of an organic anion that is an organic compound possessing at least one carboxylic acid function in the carboxylate form or at least one sulfonic acid function in the sulfonate form or at least one sulfate function. According to this particular aspect, the invention relates to a composition (C₁) as defined above, characterized in that the salt (S) is an organic salt consisting of a monovalent or multivalent metal cation more particularly selected from the elements of the group consisting of the cations of sodium, potassium, lithium, calcium, magnesium, zinc, manganese, iron, copper, cobalt, silver, gold, aluminum, barium, bismuth, selenium, zirconium, strontium and tin. According to this particular aspect, the salt (S) is an organic salt consisting of the cation selected from the elements of the group consisting of the cations of sodium, calcium, magnesium, zinc and manganese, and even more particularly the salt (S) is an organic salt consisting of the sodium cation.

According to a particular aspect, the invention relates to a composition (C₁) as defined above, characterized in that the salt (S) is an organic salt consisting of a cation, which is the ammonium ion or a metal cation as described above, and of an organic anion which is an organic compound possessing at least one carboxylic acid function in the carboxylate form selected from the elements of the group consisting of glycolic acid, citric acid, tartaric acid, salicylic acid, lactic acid, mandelic acid, ascorbic acid, pyruvic acid, fumaric acid, retinoic acid, benzoic acid, kojic acid, malic acid, gluconic acid, galacturonic acid, propionic acid, heptanoic acid, 4-aminobenzoic acid, cinnamic acid, benzalmalonic acid, aspartic acid and glutamic acid.

According to an even more particular aspect, the invention relates to a composition (C₁) as defined above, characterized in that the salt (S) is an organic salt selected from the elements of the group consisting of sodium glycolate, sodium citrate, sodium salicylate, sodium lactate, sodium gluconate, zinc gluconate, manganese gluconate, copper gluconate and magnesium aspartate.

According to another particular aspect, the invention relates to a composition (C₁) as defined above, characterized in that the salt (S) is an organic salt consisting of a cation, which is the ammonium ion or a metal cation as described above, and of an organic anion which is an organic compound possessing at least one sulfonic acid function in the sulfonate form selected from the elements of the group consisting of 2-phenylbenzimidazole-5-sulfonic acid, the sulfonic acids derived from the benzophenones, for example 4-hydroxy 2-methoxy 5-(oxo-phenylmethyl) benzenesulfonic acid (said acid being registered under the designation Benzophenone-4), the sulfonic acids derived from 3-benzylidene camphor, for example 4-(2-oxo 3-bornylidenemethyl) benzenesulfonic acid or 2-methyl 5-(2-oxo-3-bornylidenemethyl) benzenesulfonic acid.

According to an even more particular aspect, the invention relates to a composition (C₁) as defined above, characterized in that the salt (S) is an organic salt selected from the elements of the group consisting of sodium 2-phenylbenzimidazole-5-sulfonate and sodium 4-hydroxy 2-methoxy 5-(oxo-phenylmethyl) benzenesulfonate.

2-Phenylbenzimidazole-5-sulfonic acid is marketed notably under the brand name EUSOLEX™232 by the company Merck. Sodium 4-hydroxy 2-methoxy 5-(oxophenylmethyl) benzenesulfonate is registered under the designation benzophenone-5.

In general, composition (C₁) according to the present invention comprises, in addition to said fatty phase (P₁) of the stabilizing system made up of the combination in situ of the crosslinked anionic polyelectrolyte (AP) and galactomannan (GM) as defined above, and of said cosmetically acceptable aqueous phase (P₂) as defined above, adjuvants and/or additives usually employed in the area of cosmetic, dermocosmetic, pharmaceutical or dermo-pharmaceutical formulations. Among the additives that may be present in the compositions (C₁) according to the present invention, we may mention film-forming compounds, hydrotropes, plasticizers, opacifiers, luster agents, superfatting agents, sequestering agents, chelating agents, nonionic detergent surfactants, antioxidants, perfumes, preservatives, conditioners, bleaching agents intended for bleaching body hair and the skin, active principles intended to provide a treating action with respect to the skin or the hair, mineral fillers or pigments, particles providing a visual effect or intended for encapsulation of active ingredients, exfoliating particles, texture agents, optical brighteners, insect repellents.

Among the opacifiers and/or luster agents that may be combined with composition (C₁) according to the present invention, we may mention in particular the palmitates, the stearates or the hydroxystearates of sodium or of magnesium, the monostearates or distearates of ethylene or of polyethylene glycol, the fatty alcohols, the homopolymers and copolymers of styrene such as the styrene acrylate copolymer marketed under the name MONTOPOL™ OP1 by the company SEPPIC.

Among the texture agents that may be combined with composition (C₁) according to the present invention, we may mention the N-acyl derivatives of amino acids, such as lauroyl lysine marketed under the name AMINOHOPE™LL by the company AJINOMOTO, octenyl starch succinate marketed under the name DRYFLO™ by the company NATIONAL STARCH, myristyl polyglucoside marketed by SEPPIC under the name MONTANOV™ 14, cellulose fibers, cotton fibers, chitosan fibers, talc, sericite, mica.

Among the active principles that may be combined with composition (C₁) according to the present invention, we may mention for example vitamins and derivatives thereof, notably esters thereof, such as retinol (vitamin A) and its esters (retinyl palmitate for example), ascorbic acid (vitamin C) and its esters, the sugar derivatives of ascorbic acid (for example ascorbyl glucoside), tocopherol (vitamin E) and its esters (for example tocopherol acetate), vitamin B3 or B10 (nicotinamide and its derivatives); compounds displaying a lightening or depigmenting action on the skin, for example SEPIWHITE™MSH, arbutin, kojic acid, hydroquinone, VEGEWHITE™, GATULINE™, SYNERLIGHT™, BIOWHITE™, PHYTOLIGHT™, DERMALIGHT™, CLARISKIN™ MELASLOW™, DERMAWHITE™, ETHIOLINE, MELAREST™, GIGAWHITE™, ALBATINE™, LUMISKIN™; compounds displaying a soothing action such as SEPICALM™ S, allantoin and bisabolol; anti-inflammatory agents; compounds displaying a hydrating action, for example urea, the hydroxyureas, glycerol, the polyglycerols, AQUAXYL™, glycerolglucoside; the extracts of polyphenols for example grape extracts, pine extracts, wine extracts, olive extracts; compounds displaying a slimming or lipolytic action such as caffeine or derivatives thereof, ADIPOSLIM™, ADIPOLESS™; the N-acylated proteins; the N-acylated peptides, for example MATRIXIL™; the N-acylated amino acids; the partial hydrolyzates of N-acylated proteins; amino acids; peptides; the total hydrolyzates of proteins; soybean extracts, for example Raffermine™ wheat extracts for example TENSINE™ or GLIADINE™; vegetable extracts, such as vegetable extracts rich in tannins, vegetable extracts rich in isoflavones or vegetable extracts rich in terpenes; extracts of fresh-water or marine algae; marine extracts in general such as coral; essential waxes; bacterial extracts; ceramides; phospholipids; compounds displaying an antimicrobial action or a purifying action, for example LIPACIDE™ C8G, LIPACIDE™ UG, SEPICONTROL™ A5; OCTOPIROX™ or SENSIVA™ SC50; compounds displaying an energizing or stimulating property such as Physiogenyl™ panthenol and derivatives thereof such as SEPICAP™ MP; antiaging active ingredients such as SEPILIFT™ DPHP, LIPACIDE™ PVB, SEPIVINOL™, SEPIVITAL™, MANOLIVA™, PHYTO-AGE™, TIMECODE™; SURVICODE™; antiphoto-aging active ingredients; active ingredients protecting the integrity of the dermal-epidermal junction; active ingredients increasing the synthesis of components of the extracellular matrix, for example collagen, the elastins, the glycosaminoglycans; active ingredients acting favorably on cellular communication—chemical such as the cytokines or physical such as the integrins; active ingredients creating a sensation of “heating” on the skin such as the activators of the microcirculation of the skin (for example derivatives of nicotinic acid) or products creating a sensation of “freshness” on the skin (for example menthol and derivatives); active ingredients improving the microcirculation of the skin, for example veinotonic agents; draining active ingredients; active ingredients with a decongestant purpose, for example extracts of Ginkgo biloba, ivy, horse chestnut, bamboo, Ruscus, butchers broom, Centella asiatica, fucus, rosemary, willow.

Among the active principles that may be combined with composition (C₁) according to the present invention, we may mention more particularly the skin bronzing or tanning agents, for example dihydroxyacetone, isatin, alloxan, ninhydrin, glyceraldehyde, mesotartaric aldehyde, glutaraldehyde, erythrulose.

Among the nonionic detergent surfactants that may be combined with composition (C₁) according to the present invention, we may mention the ethoxylated derivatives of fatty alcohols comprising from 8 to 12 carbon atoms, the ethoxylated derivatives of fatty acids comprising from 8 to 12 carbon atoms, the ethoxylated derivatives of fatty esters comprising from 8 to 12 carbon atoms, the ethoxylated derivatives of monoglycerides comprising from 8 to 12 carbon atoms, the alkylpolyglycosides of formula (II):

R2-O—(S)y-H (II)

in which y represents a decimal number between 1 and 5, S represents the residue of a reducing sugar and R2 represents a linear or branched, saturated or unsaturated alkyl radical, having from 5 to 16 carbon atoms, preferably from 8 to 14 carbon atoms, or a mixture of compounds of formula (II).

The nonionic detergent surfactants that may be combined with composition (C₁) according to the present invention are more particularly selected from the elements of the group consisting of the caprylyl capryl glucosides, marketed notably under the brand name ORAMIXTMCG 110 by the company SEPPIC, decylglucoside, marketed notably under the brand name ORAMIX™NS 10 by the company SEPPIC.

Among the pigments that may be combined with composition (C₁) according to the present invention, we may mention titanium dioxide, brown iron oxides, yellow iron oxides, black iron oxides, or red iron oxides, or white or colored nacreous pigments such as titanium mica.

Among the sun filters that may be combined with composition (C₁) according to the present invention, we may mention all those given in the amended cosmetic directive 76/768/EEC annex VII, for example titanium oxide, zinc oxide, the esters of cinnamic acid, for example 2-ethylhexyl 4-methoxycinnamate, isopentyl 4-methoxycinnamate, the nonionic derivatives of benzophenone, the esters of 4-aminobenzoic acid, for example 2-ethylhexyl 4-(dimethylamino)benzoate, amyl 4-(dimethylamino)benzoate.

According to another particular aspect, the invention relates to a composition (C₁) as defined above, characterized in that its dynamic viscosity measured at a temperature of 20° C., using a viscosimeter of the Brookfield type, is greater than or equal to 30 000 mPa·s and less than or equal to 200 000 mPa·s, more particularly greater than or equal to 40 000 mPa·s and less than or equal to 130 000 mPa·s, and even more particularly greater than or equal to 50 000 mPa·s and less than or equal to 130 000 mPa·s.

When the dynamic viscosity of composition (C₁) is less than or equal to about 100 000 mPa·s at a temperature of 20° C., said dynamic viscosity is measured using a viscosimeter of the Brookfield LVT type at a speed of 6 revolutions per minute.

When the dynamic viscosity of composition (C₁) is above about 100 000 mPa·s at a temperature of 20° C., said dynamic viscosity is measured using a viscosimeter of the Brookfield RVT type at a speed of 5 revolutions per minute.

According to a particular aspect, the invention relates to a composition (C₁) as defined above, characterized in that the conductivity of said composition (C₁), measured at a temperature of 20° C. with a conductivity meter of type LF 196 from the company WTW equipped with a Tetracon 96 electrode, is greater than or equal to 15 millisiemens·cm⁻¹ (mS·cm⁻¹) and less than or equal to 200 mS·cm⁻¹, more particularly greater than or equal to 15 mS·cm⁻¹ and less than or equal to 150 mS·cm⁻¹.

Composition (C₁) according to the present invention is notably in the form of an emulsion or of a microemulsion with an aqueous continuous phase.

When composition (C₁) according to the present invention has suitable fluidity characteristics, it may also serve for impregnation of substrates consisting of synthetic or natural textile fibers, woven or nonwoven, or of papers, for constituting articles, for example wipes, intended for the care, protection or cleaning of the skin, scalp or hair, or for example papers for sanitary or domestic use.

Composition (C₁) according to the present invention may be used by application on the skin, the mucosae, the hair or the scalp, either direct application in the case of a cosmetic, dermocosmetic, dermopharmaceutical or pharmaceutical composition, or indirect application in the case of a product for care, protection, or cleaning of the body, in the form of a textile article, for example a wipe, or a paper article, for example paper for sanitary use, intended to be in contact with the skin, hair or scalp.

Composition (C₁) as defined above according to the present invention is stable over time after a period of storage of at least one month at 20° C. and retains a homogeneous appearance, and does not display appearance of masses or lumps, at the end of this same period of storage in the same experimental conditions, without necessitating the incorporation of emulsifying surfactants in said composition (C₁).

According to a particular aspect, the present invention relates to a composition (C₁) comprising for 100% of its weight, an amount of 0 wt % of emulsifying surfactants (EM) selected from the elements of group (G₁) consisting of:
fatty acids comprising from 14 to 22 carbon atoms,
ethoxylated fatty acids comprising from 14 to 22 carbon atoms,
esters of fatty acid comprising from 14 to 22 carbon atoms and of sorbitol,
esters of fatty acid comprising from 14 to 22 carbon atoms and of polyglycerol,
ethoxylated fatty alcohols comprising from 14 to 22 carbon atoms,
esters of fatty acid comprising from 14 to 22 carbon atoms and of sucrose,
alkylpolyglycosides of formula (II):

R₃—O—(S)_{z}—H (III)

in which z represents a decimal number between 1 and 5, S represents the residue of a reducing sugar and R₃ represents a linear or branched, saturated or unsaturated alkyl radical, having from 14 to 22 carbon atoms, preferably from 16 to 22 carbon atoms, or a mixture of compounds of formula (III).

In the definition of formula (III) as defined above, z is a decimal number representing the average degree of polymerization of residue S. When z is an integer, (S)_{z} is the polymer residue of rank z of residue S. When z is a decimal number, formula (III) represents a mixture of compounds:

a1 R₃—O—S—H+a2 R₃—O—(S)₂—H+a3 R₃—O—(S)₃—H+ . . . +aq R₃—O—(S)_{q}—H with q representing an integer between 1 and 10 and in the molar proportions a1, a2, a3, . . . aq such that:
q=1
Σaq=1; a1>0
q=10

In formula (III) as defined above, z is between 1.05 and 5.0 and more particularly between 1.05 and 2.

In formula (III) as defined above, R₃ represents for example the n-tetradecyl radical, the n-hexadecyl radical, the n-octadecyl radical, the n-eicosyl radical, the n-dodecosyl radical.

“Reducing sugar” denotes, in the definition of formula (III), the saccharide derivatives that do not have, in their structures, a glycosidic bond established between an anomeric carbon and the oxygen of an acetal group as defined in the reference work “Biochemistry”, Daniel Voet/Judith G. Voet, p. 250, John Wiley & Sons, 1990. The oligomeric structure (S)_{z} may be in any form of isomerism, whether it is optical isomerism, geometric isomerism or positional isomerism; it may also represent a mixture of isomers.

In formula (III) as defined above, the group R₃—O— is bound to S by the anomeric carbon of the saccharide residue, so as to form an acetal function.

In formula (III) as defined above, S represents the residue of a reducing sugar selected from glucose, xylose or arabinose.

According to another particular aspect, the present invention relates to a composition (C₁) comprising, for 100% of its weight, from 0.1 to 10 wt %, more particularly from 0.1 to 5 wt %, and even more particularly from 0.5 to 3 wt % of at least one emulsifying surfactant (EM) selected from the elements of group (G₁) as defined above.

According to this other particular aspect, the weight ratio between the sum of the amount by weight of anionic polyelectrolyte (AP) and the amount by weight of galactomannan (GM), as defined above, and the amount by weight of the emulsifier (EM) is greater than or equal to 1.0, more particularly greater than or equal to 5.0, and even more particularly greater than or equal to 10.0.

According to another aspect, the present invention relates to a method of preparing composition (C₁) as defined above, characterized in that it comprises:

At least one step a) of preparing a phase (P′₁) by mixing the crosslinked anionic polyelectrolyte (AP) and galactomannan (GM) in the fatty phase (P₁); and

At least one step b) of emulsifying the phase (P′₁) obtained at the end of step a) with the cosmetically acceptable aqueous phase (P₂).

In the method according to the invention, the fatty phase (P₁) comprises one or more oils and/or one or more waxes as defined above.

In the case when the fatty phase (P₁) does not consist of a single oil or a single wax, the fatty phase (P₁) is prepared by mixing its constituent ingredients at a temperature typically between 20° C. and 85° C., and even more particularly at a temperature between 20° C. and 60° C., and by means of any mixing device known by a person skilled in the art, for example by means of a mechanical stirring device equipped with a rotor of the “anchor” type, at stirring speeds between 50 revolutions per minute and 500 revolutions per minute, more particularly between 50 revolutions per minute and 300 revolutions per minute.

In the method according to the invention, step a) of preparing a phase (P′₁) by mixing the crosslinked anionic polyelectrolyte (AP) and galactomannan (GM) in the fatty phase (P₁) may advantageously be carried out at a temperature less than or equal to 85° C. and greater than or equal to 20° C., more particularly at a temperature less than or equal to 60° C. and greater than or equal to 20° C.

In the method according to the invention, step a) of preparing a phase (P′₁) by mixing the crosslinked anionic polyelectrolyte (AP) and galactomannan (GM) in the fatty phase (P1) may be carried out by means of any mixing device known by a person skilled in the art, for example by means of a mechanical stirring device equipped with a rotor of the “anchor” type, at stirring speeds between 50 revolutions per minute and 500 revolutions per minute, more particularly between 50 revolutions per minute and 300 revolutions per minute, and for example by means of a stirring device of the rotor-stator type at stirring speeds between 100 revolutions per minute and 10 000 revolutions per minute, more particularly between 500 revolutions per minute and 4000 revolutions per minute.

In the method according to the invention, step b) of emulsifying the phase (F₁) obtained at the end of step a) with the aqueous phase (P₂) may advantageously be carried out at a temperature between 20° C. and 90° C., more particularly at a temperature between 20° C. and 85° C., and even more particularly at a temperature between 20° C. and 60° C.

In the method according to the invention, step b) of emulsifying the phase (F₁) obtained at the end of step a) with the aqueous phase (P₂) may be carried out by means of any mixing device known by a person skilled in the art, for example by means of a mechanical stirring device equipped with a rotor of the “anchor” type, at stirring speeds between 50 revolutions per minute and 500 revolutions per minute, more particularly between 50 revolutions per minute and 300 revolutions per minute, and for example by means of a stirring device of the rotor-stator type at stirring speeds between 100 revolutions per minute and 10 000 revolutions per minute, more particularly between 500 revolutions per minute and 4000 revolutions per minute.

In the method according to the invention, the cosmetically acceptable aqueous phase (P₂) comprises water, and optionally one or more cosmetically acceptable organic solvents as described above, and from 1 to 25 wt %, for 100% of the weight of said cosmetically acceptable aqueous phase (P₂), of at least one salt (S) in dissolved form and as defined above.

The cosmetically acceptable aqueous phase (P₂) is prepared by mixing water, and optionally one or more cosmetically acceptable organic solvents, with at least one salt (S) as described above, at a temperature between 20° C. and 85° C., and even more particularly at a temperature between 20° C. and 60° C., and by means of any mixing device known by a person skilled in the art, for example by means of a mechanical stirring device equipped with a rotor of the “anchor” type, at stirring speeds between 50 revolutions per minute and 500 revolutions per minute, more particularly 20 between 50 revolutions per minute and 300 revolutions per minute.

According to another aspect, the present invention relates to the cosmetic use of composition (C₁) as defined above, for cleaning, for protection and/or for care of the skin, hair, scalp or mucosae.

In the context of the present invention, “cosmetic use” denotes the uses of composition (C₁) intended to improve and/or preserve the outward aesthetic appearance of the skin, hair, scalp or mucosae.

According to a more particular aspect, composition (C₁) according to the present invention may be used for cleaning the skin, mucosae, hair or scalp, and more particularly may be used as bath or shower gel, or as shampoo. In this particular use, it further comprises at least one nonionic detergent surfactant as described above.

According to another more particular aspect, composition (C₁) according to the present invention may be used for care or protection of the skin, for example as cream, as milk or as lotion for care or for protection of the face, hands and body.

According to this particular aspect, composition (C₁) according to the present invention may also be used more particularly as a product for protecting the skin against the sun's rays, as a product for makeup of the skin, as a product protecting the skin against cutaneous aging, as a product for hydrating the skin, as a product for cosmetic treatment of acne and/or black spots and/or comedones.

The following examples illustrate the invention but without limiting it.

### 1-1 Preparation of a terpolymer of ammonium 2-methyl 2-[(1-oxo 2-propeny)amino] 1-propanesulfonate, of N,N-dimethylacrylamide and of tetraethoxylated laurel methacrylate [AMPSNH₄/DMAM/MAL(4OE) 77.4/19.2/3.4 molar], crosslinked with trimethylol propanetriacrylate (TMPTA)

### Example According to the Invention

A reactor, maintained at 25° C. with stirring, is charged with 592 g of a 15 wt % aqueous solution of ammonium 2-methyl 2-[(1-oxo 2-propenyl)amino] 1-propanesulfonate (AMPSNH₄) in a tert-butanol/water mixture (97.5/2.5 by volume), 10.1 g of N,N-dimethylacrylamide (DMAM), 4.2 g of tetraethoxylated lauryl methacrylate [MAL(4OE)] and 0.75 g of TMPTA.

After a sufficient time to achieve good homogenization of the solution, the latter is deoxygenated by bubbling with nitrogen heated to 70° C. 0.42 g of dilauroyl peroxide is then added and the reaction mixture is then held for about 60 minutes at 70° C. and then for 2 hours at 80° C.

After cooling, the powder that formed during polymerization is filtered and dried, obtaining the desired product, called hereinafter: “Polyelectrolyte PA₁”.

### 2-1: Preparation of Oil-in-Water Emulsions According to the Invention

Six oil-in-water emulsions according to the invention are prepared, designated (E₁) to (E₆), for which the proportions by weight of their constituents are given in Table 1 below, using the following procedure:
In a first beaker, the polyelectrolyte PA₁ and guar gum are dispersed in a fatty phase gradually and successively at a temperature of 20° C., with mechanical stirring at 80 revolutions per minute.
The aqueous phase comprising water and the desired amount of salt by weight is prepared in a second beaker, at a temperature of 20° C.
The contents of the first beaker are poured gradually into the second beaker at a temperature of 20° C., with mechanical stirring by means of a deflocculator at 1200 revolutions per minute.
The mixture obtained is stirred for 10 minutes, and then discharged, obtaining the oil-in-water emulsions (E₁) to (E₆).

TABLE 1

| Emulsion | (E₁) | (E₂) | (E₃) | (E₄) | (E₅) | (E₆) |
|---|---|---|---|---|---|---|
| Fatty phase: | | | | | | |
| C8-C10 triglycerides | 15% | 15% | 15% | 15% | 15% | 15% |
| Stabilizing system: | | | | | | |
| Polyelectrolyte (PA₁) | 1% | 1% | 1% | 1.25% | 0.5% | 1.50% |
| Guar gum⁽²⁾ | 1% | 1% | 1% | 0.75% | 1.50% | 0.50% |
| Aqueous phase: | | | | | | |
| Water | Qs. 100% | Qs. 100% | Qs. 100% | Qs. 100% | Qs. 100% | Qs. 100% |
| Sodium chloride | 2% | 4% | 6% | 8% | 10% | 2% |
| Geogard ™ 221⁽¹⁾ | 0.6% | 0.6% | 0.6% | 0.6% | 0.6% | 0.6% |

⁽¹⁾Geogard ™ 221 is a mixture of dehydroacetic acid and benzyl alcohol used as a preservative and marketed by the company LONZA.
⁽²⁾Guar gum (CAS number: 9000-30-0) is marketed under the name “VIDOCHREM ™ E” by the company UNIPEKTIN Ingredients AG.

### 2-2: Preparation of Oil-in-Water Emulsions According to the Prior Art

Six oil-in-water emulsions are prepared, designated (F₁) to (F₆), for which the proportions by weight of their constituents are given in Table 2 below, using the following procedure:
In a first beaker, the polyelectrolyte PA₁ and guar gum are dispersed in a fatty phase gradually and successively at a temperature of 20° C. with mechanical stirring at 80 revolutions per minute.
The aqueous phase comprising water and the desired amount of salt by weight is prepared in a second beaker, at a temperature of 20° C.
The contents of the first beaker are poured gradually into the second beaker at a temperature of 20° C., with mechanical stirring by means of a deflocculator at 1200 revolutions per minute.
The mixture obtained is stirred for 10 minutes, and then discharged, obtaining the oil-in-water emulsions (F₁) to (F₆).

TABLE 2

| Emulsion | (F₁) | (F₂) | (F₃) | (F₄) | (F₅) | (F₆) |
|---|---|---|---|---|---|---|
| Fatty phase: | | | | | | |
| C8-C10 triglycerides | 15% | 15% | 15% | 15% | 15% | 15% |
| Stabilizing system: | | | | | | |
| Polyelectrolyte (PA₁) | 1% | 2% | 2% | 0% | 1.25% | 0.5% |
| Guar gum | 1% | 0% | 0% | 2% | 0.75% | 1.50% |
| Aqueous phase: | | | | | | |
| Water | Qs. 100% | Qs. 100% | Qs. 100% | Qs. 100% | Qs. 100% | Qs. 100% |
| Sodium chloride | 0% | 2% | 4% | 4% | 0% | 0% |
| Geogard ™ 221 | 0.6% | 0.6% | 0.6% | 0.6% | 0.6% | 0.6% |

### 2-3: Determination of the Properties and Characteristics of the Oil-in-Water Emulsions According to the Invention Compared with the Oil-in-Water Emulsions of the Prior Art

The oil-in-water emulsions (E₁) to (E₆) according to the invention and the oil-in-water emulsions (F₁) to (F₆) according to the prior art, thus prepared, are then stored in an insulated climate chamber controlled at a temperature of 20° C. for 7 days. At the end of this period of 7 days and for each oil-in-water emulsion:
The visual appearance is observed.
The dynamic viscosity (p) of each emulsion is measured at 20° C., using a viscosimeter of the Brookfield LVT type, equipped with a suitable rotor, at a speed of 6 revolutions per minute (V6) when said dynamic viscosity is less than or equal to about 100 000 mPa·s, or using a viscosimeter of the Brookfield RVT type, equipped with a suitable rotor, at a speed of 5 revolutions per minute when said dynamic viscosity is above 100 000 mPa·s.
The conductivity is measured at 20° C., with a conductivity meter of type LF 196 from the company WTW equipped with a Tetracon™ 96 electrode.

The oil-in-water emulsions are then replaced and stored in the same insulated climate chamber controlled at a temperature of 20° C. for up to three months. After a period of three months, each emulsion is taken out of the climate chamber to examine its appearance. The results obtained for the oil-in-water emulsions (E₁) to (E₆) according to the invention are given in Table 3 below and the results obtained for the comparative oil-in-water emulsions (F₁) to (F₆) are given in Table 4 below.

TABLE 3

| Emulsion | (E₁) | (E₂) | (E₃) | (E₄) | (E₅) | (E₆) |
|---|---|---|---|---|---|---|
| Visual appearance after | ++ | ++ | ++ | ++ | ++ | ++ |
| 7 days at 20° C. | | | | | | |
| Viscosity (Brookfield LVT, | 59 000 | 51 400 | 57 000 | 43 000 | 73 500 | 41 000 |
| V6) in mPa · s | | | | | | |
| Conductivity in mS · cm⁻¹ | 32.3 | 57.5 | 118.7 | 58.2 | 56.6 | 58.0 |
| Visual appearance after | ++ | ++ | ++ | ++ | ++ | ++ |
| 3 months at 20° C. | | | | | | |

++: Appearance homogeneous and smooth

TABLE 4

| Emulsion | (F₁) | (F₂) | (F₃) | (F₄) | (F₅) | (F₆) |
|---|---|---|---|---|---|---|
| Visual appearance after 7 days at | (—) | (—) | (—) | (—) | (—) | (—) |
| 20° C. | | | | | | |
| Viscosity (Brookfield LVT, V6) in | 175 000* | 72 500 | 55 000 | 32 000 | 83 500 | 89 000 |
| mPa · s | | | | | | |
| Conductivity in mS · cm⁻¹ | 1.4 | 31.6 | 57.0 | 55.4 | 1.7 | 0.7 |
| Visual appearance after 3 months | (—) | (—) | (—) | (—) | (—) | (—) |
| at 20° C. | | | | | | |

*: dynamic viscosity measured at 20° C. with the Brookfield RVT viscosimeter, speed 5 revolutions per minute.
(-): Presence of masses and lumps
(--): Heterogeneous appearance with presence of masses and lumps

### 2-4: Analysis of the Results

The results are judged satisfactory when the visual appearance of an oil-in-water emulsion is judged homogeneous and smooth after storage of said oil-in-water emulsion for three months at 20° C., and when its dynamic viscosity measured at 20° C., using a viscosimeter of the Brookfield LVT type at a speed of 6 revolutions per minute, equipped with a suitable rotor, is greater than or equal to 30 000 mPa·s.

Emulsions (E₁) to (E₆) according to the invention are of a smooth appearance, without masses and lumps, even after prolonged storage for 3 months at 20° C.

The results obtained with emulsions (F₂) and (F₃) reveal that when the stabilizing system of the oil-in-water emulsion consists of the polyelectrolyte (PA₁) only, in the presence of an amount of 2% and of 4% of sodium chloride, oil-in-water emulsions with a homogeneous and smooth appearance are not obtained after a period of storage of 7 days at 20° C.

Moreover, the results obtained with emulsion (F₄) reveal that when the stabilizing system of the oil-in-water emulsion consists of guar gum only, in the presence of 4% of NaCl, oil-in-water emulsions with a homogeneous and smooth appearance are not obtained after a period of storage of 7 days at 20° C.

For weight ratios of guar gum to polyelectrolyte (PA₁) equal to 1/1, ⅗ and 3/1 respectively and in the absence of sodium chloride, the results obtained with emulsions (F₁), (F₅) and (F₆) reveal that oil-in-water emulsions with a homogeneous and smooth appearance are not obtained after a period of storage of 7 days at 20° C.

Comparison of the results obtained with oil-in-water emulsions (E₁) to (E₆) according to the invention against those obtained with oil-in-water emulsions (F₁) to (F₆) according to the prior art shows an improvement in the appearance of the salt-rich oil-in-water emulsions, while maintaining a high level of viscosity, constituting an additional technical effect due to the invention according to the present patent application.

### Examples of Formulas for Purposes of Illustration

### 3-1: Body Care Hydrating Gel-Creme

### Formula:

| | | |
|---|---|---|
| A | Jojoba oil | 14.10% |
| | C12-C15 alkyl benzoate | 6.7% |
| | DC 245 | 4.2% |
| | DL alpha Tocopherol | 0.05% |
| B | Maris Aqua | 70.85% |
| | AQUAXYL ™ | 3% |
| C | Polyelectrolyte (PA₁) | 2% |
| | Guar gum⁽²⁾ | 1% |
| D | Euxyl ™ PE9010 | 1% |
| | Fragrance | 0.1% |

### Procedure:

Mix the constituents of fatty phase A at a temperature of 80° C. with stirring. Then add the ingredients of phase C successively.

Prepare aqueous phase B and heat it to 80° C. with stirring.

Add aqueous phase B gradually to the mixture of phases A+C and then emulsify using a stirrer equipped with a Silverson rotor-stator.

Then cool to 25° C., and then add phase D.

### 3-2: Gel-Creme Face Mask

### Formula:

| | | |
|---|---|---|
| A | Triglycerides 4555 (C8C10) | 9% |
| | C12-C15 alkyl benzoate | 4% |
| | Isohexadecane | 2% |
| | DL alpha Tocopherol | 0.10% |
| B | Maris Aqua | q.s. 100% |
| C | Polyelectrolyte (PA₁) | 1.3% |
| | Guar gum⁽²⁾ | 0.7% |
| D | Euxyl PE9010 | 1% |
| | Fragrance | 0.1% |

### Procedure:

Mix the constituents of fatty phase A at a temperature of 80° C. with stirring. Then add the ingredients of phase C successively.

Prepare aqueous phase B and heat it to 80° C. with stirring.

Add aqueous phase B gradually to the mixture of phases A+C and then emulsify using a stirrer equipped with a Silverson rotor-stator. R

Then cool to 25° C. and add phase D.

### 3-3: Body Cream

### Formula:

| | |
|---|---|
| Triglycerides 4555 (C8C10) | 12% |
| C12-C15 alkyl benzoate | 5.3% |
| Isohexadecane | 2.7% |
| Cetyl alcohol | 2% |
| DL alpha Tocopherol | 0.10% |
| Polyelectrolyte (PA₁) | 1.5% |
| Guar gum⁽²⁾ | 0.5% |
| Water | q.s. 100% |
| Givobio ™ GZn | 1% |
| Sepicalm ™ S | 3% |
| Euxyl ™ PE9010 | 1% |
| Fragrance | 0.1% |

### 3-4: Organo-Mineral Sun Spray

### Formula:

| | | |
|---|---|---|
| A | Isodecyl neopentanoate | 20% |
| | Cyclodimethicone | 5% |
| | Ethylhexylmethoxicinnamate | 6% |
| | Butyl Methoxydibenzoylmethane | 3% |
| | DL alpha Tocopherol | 0.05% |
| B | Water | q.s. 100% |
| | EDTA tetrasodium | 0.2% |
| | Glycerin | 7% |
| | Phenyl Benzyimidazole Sulfonic Acid | 3% |
| | (salified with the necessary molar amount of soda) | |
| C | Polyelectrolyte (PA₁) | 1.3% |
| | Guar gum⁽²⁾ | 0.7% |
| D | SEPICIDE ™ HB | 1% |
| | Fragrance | 0.1% |

AQUAXYL™ (INCI name: Xylitylglucoside & Anhydroxylitol & Xylitol): Hydrating composition marketed by the company SEPPIC.
Euxyl™ PE9010 (INCI name: Phenoxyethanol & Ethylhexyl Glycerin): Composition used as preservative.
GIVOBIO™ GZn (INCI name: Zinc Gluconate): Composition marketed by the company SEPPIC.
LANOL™ 99 (INCI name: Isononyl Isononanoate): Ester used as oily phase in the preparation of cosmetic compositions and distributed by the company SEPPIC.
Maris Aqua: seawater at 8% sodium chloride.
SEPICIDE™ HB (INCI name: Phenoxyethanol/Methylparaben/Ethylparaben/Propylparaben/Butylparaben): Preservative containing phenoxyethanol, marketed by the company SEPPIC.
SEPICALM™ S: (INCI name: Sodium Cocoyl Aminoacids And Sarcosine And Potassium Aspartate And Magnesium Aspartate): anti-inflammatory composition marketed by the company SEPPIC.
SERENIKS™ 207 (INCI name: Tsuga Canadensis Leaf Extract And Water And Butylene Glycol) is an antiaging composition.

1. A composition (C₁) in the form of an oil-in-water type emulsion, comprising, for 100% of its the composition's weight:
from 5 to 55 wt % of a fatty phase (P₁) consisting of at least one oil and/or at least one wax,
from 0.025 to 3.75 wt % of at least one crosslinked anionic polyelectrolyte (AP) resulting from the polymerization of at least one monomer possessing a partially or totally salified strong acid function, with at least one neutral monomer, and at least one monomer of formula (I): 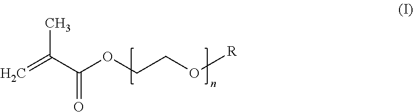
in which R represents a linear or branched alkyl radical comprising from eight to twenty carbon atoms and n represents a number greater than or equal to one and less than or equal to twenty, in the presence of at least one crosslinking agent,
from 0.025 to 3.75 wt % of at least one galactomannan (GM) having a degree of substitution (DS) of about 1/1 or of about ½ or of about ¼ or of about ⅕,
from 37.5 to 94.95 wt % of a cosmetically acceptable aqueous phase (P₂), said aqueous phase (P₂) comprising, for 100% of its weight, from 1 to 25 wt % of at least one salt (S) in dissolved form,
wherein the weight ratio of the galactomannan (GM) to the crosslinked anionic polyelectrolyte (AP) is greater than or equal to ⅓ and less than or equal to 3/1.

2. The composition (C₁) as defined in claim 1, wherein said galactomannan (GM) has a degree of substitution (DS) of about ½.

3. The composition (C₁) as defined in claim 1, wherein said crosslinked anionic polyelectrolyte (AP) comprises, for 100 mol % of its constituent monomers:
from 20 mol % to 80 mol % of monomer units derived from a monomer comprising a partially or totally salified strong acid function;
from 15 mol % to 75 mol % of monomer units derived from a neutral monomer;
from 0.5 to 5 mol % of monomer units derived from a monomer of formula (I) as defined above.

4. The composition (C₁) as defined in claim 1, wherein in said crosslinked anionic polyelectrolyte (AP), said monomer possessing a strong acid function is partially salified or totally salified 2-methyl 2-[(1-oxo 2-propenyl)amino] 1-propanesulfonic acid.

5. The composition (C₁) as defined in claim 1, wherein in said crosslinked anionic polyelectrolyte (AP), said neutral monomer is selected from acrylamide, (2-hydroxyethyl)acrylate or N,N-dimethylacrylamide.

6. The composition (C₁) as defined in claim 1, wherein in said crosslinked anionic polyelectrolyte (AP), said monomer of formula (I) is tetraethoxylated lauryl methacrylate.

7. The composition (C₁) as defined in claim 1, wherein said crosslinked anionic polyelectrolyte (AP) is a terpolymer of 2-methyl 2-[(1-oxo 2-propenyl)amino] 1-propanesulfonic acid partially salified in the form of ammonium salt, of N,N-dimethylacrylamide and of tetraethoxylated lauryl methacrylate, crosslinked with trimethylol propanetriacrylate.

8. The composition (C₁) as defined in claim 1, wherein the salt (S) is an inorganic salt consisting of a cation, which is the ammonium ion or a metal cation, and of an anion selected from the elements of the group consisting of the halides, carbonates, bicarbonates, phosphates, nitrates, borates and sulfates.

9. The composition (C₁) as defined in claim 1, wherein the salt (S) is an organic salt consisting of a cation, which is the ammonium ion or a metal cation, and of an organic anion which is an organic compound possessing at least one carboxylic acid function in the carboxylate form or at least one sulfonic acid function in the sulfonate form or at least one sulfate function.

10. The composition (C₁) as defined in claim 9, wherein the salt (S) is an organic salt selected from the elements of the group consisting of sodium glycolate, sodium citrate, sodium salicylate, sodium lactate, sodium gluconate, zinc gluconate, manganese gluconate, copper gluconate and magnesium aspartate.

11. The composition (C₁) as defined in claim 9, wherein the salt (S) is an organic salt selected from the elements of the group consisting of sodium 2-phenylbenzimidazole-5-sulfonate and sodium 4-hydroxy 2-methoxy-5-(oxo-phenylmethyl) benzenesulfonate.

12. The composition (C₁) as defined in claim 1, wherein a dynamic viscosity of the composition measured at a temperature of 20° C., using a viscosimeter of the Brookfield type, is greater than or equal to 30 000 mPa·s and less than or equal to 200 000 mPa·s

13. A method of preparing a composition (C₁) as defined in claim 1, further comprising:
At least one step a) of preparing a phase (P′₁) by mixing the crosslinked anionic polyelectrolyte (AP) and the galactomannan (GM) in the fatty phase (P₁); and
At least one step b) of emulsifying the phase (P′₁) obtained at the end of step a) with the cosmetically acceptable aqueous phase (P₂).

14. A method for cleaning, for protection and/or for care of the skin, hair, scalp or mucosae, comprising applying an effective amount of the composition (C₁) of claim 1.

15. The composition (C₁) as defined in claim 2, wherein said crosslinked anionic polyelectrolyte (AP) comprises, for 100 mol % of its constituent monomers:
from 20 mol % to 80 mol % of monomer units derived from a monomer comprising a partially or totally salified strong acid function;
from 15 mol % to 75 mol % of monomer units derived from a neutral monomer;
from 0.5 to 5 mol % of monomer units derived from a monomer of formula (I) as defined above.

16. The composition (C₁) as defined in claim 2, wherein in said crosslinked anionic polyelectrolyte (AP), said monomer possessing a strong acid function is partially salified or totally salified 2-methyl 2-[(1-oxo 2-propenyl)amino] 1-propanesulfonic acid.

17. The composition (C₁) as defined in claim 3, wherein in said crosslinked anionic polyelectrolyte (AP), said monomer possessing a strong acid function is partially salified or totally salified 2-methyl 2-[(1-oxo 2-propenyl)amino] 1-propanesulfonic acid.

18. The composition (C₁) as defined in claim 2, wherein in said crosslinked anionic polyelectrolyte (AP), said neutral monomer is selected from acrylamide, (2-hydroxyethyl) acrylate or N,N-dimethylacrylamide.

19. The composition (C₁) as defined in claim 3, wherein in said crosslinked anionic polyelectrolyte (AP), said neutral monomer is selected from acrylamide, (2-hydroxyethyl) acrylate or N,N-dimethylacrylamide.

20. The composition (C₁) as defined in claim 4, wherein in said crosslinked anionic polyelectrolyte (AP), said neutral monomer is selected from acrylamide, (2-hydroxyethyl) acrylate or N,N-dimethylacrylamide.
